(19) United States
(12) Patent Application Publication
(10) Pub. No.: US 2022/0142534 A1
(43) Pub. Date: May 12, 2022

(54) POINT-OF-CARE DIAGNOSTIC DEVICE AND A METHOD FOR SECURE USER IDENTITY CONFIRMATION WHILE MAINTAING ANONIMITY

(71) Applicants: David Seliktar, (US); Shay Sasson, Tel Aviv (IL)

(72) Inventors: David Seliktar, Great Neck, NY (US); Shay Sasson, Tel Aviv (IL); Ilya Aronovich, Great Neck, NY (US)

(73) Assignee: Ilya Aronovich, Great Neck, NY (US)

(21) Appl. No.: 17/524,237

(22) Filed: Nov. 11, 2021

Related U.S. Application Data
(60) Provisional application No. 63/112,172, filed on Nov. 11, 2020.

(51) Int. Cl. A61B 5/15; A61B 5/157; A61B 5/1172; A61B 5/151; A61B 5/00; G01N 33/569; G16H 40/63

(52) U.S. Cl. CPC A61B 5/150763; A61B 5/157; A61B 5/150916; A61B 5/1172; A61B 5/150022; G01N 2333/165; A61B 5/742; G01N 33/56983; G16H 40/63; G01N 2469/20; A61B 5/15142

(57) ABSTRACT

A device and method for performing a diagnostic test, including determining presence or absence of antibodies to a virus. The device displays the results on a display, and includes a retractable needle and an enclosure that houses the retractable needle, a vessel for collecting or storing a bodily liquid sample collected from the subject, an enclosure containing a diluent fluid that is mixed with the collected sample, and at least one area for mixing and performing analysis.

A pressure sensor checks for pressure being applied by the test subject's finger during the test, and stops the test or disables display if it does not sense pressure of the test subject's finger during testing. A processor performs identification testing by scanning a digital fingerprint of the test subject by a digital scanner prior to evaluation, stores the data and evaluates the test results to determine whether the results exceed or reach a threshold value. The testing and evaluation are performed only after a successful identification of the test subject. The device keeps the test information and identification information about the test subject anonymous, and does not communicate the identity of the test subject and the test results to an external device.

This application claims priority of a provisional patent application No. 63/112,172, filed on Nov. 11, 2020, and entitled “A Point-of-Care Diagnostic Device and a Method for Securing the Identity of Users While Maintaining Anonymity”.

### TECHNICAL FIELD

The present invention relates to a device and a method for performing point-of-care diagnostics, such as, for example, self-testing using home-testing kit or a device that uses test subject's urine, blood, saliva or tissue to test for a medical condition such as, for example, blood sugar levels for diabetes, HIV blood test, COVID-19 antibody presence blood test, urine pregnancy test, drug presence (interactions) and many other similar types of tests that are performed outside the specialized medical lab or hospital. More specifically, the present invention relates to a device and method that preforms a medical diagnostic process at the point-of-care (without the need to send samples to the lab) using test subject's blood, blood plasma, urine, saliva, or tissue sample, and also safely and reliably confirms the unique identity of the test subject, and at the same time allows the examinee to maintain anonymity and not disclose sensitive medical information.

### BACKGROUND

The point-of-care diagnostic (POCD) testing has become very commonplace today, and it relates to medical diagnostic testing at or near the point-of-care that is, at the time and place of patient care. The term POCD also refers to self-tests that can be administered by a patient or his or her family members at home. Many POCD test kits can be operated by the patient himself or herself, and do not require medical personnel to assist with administration of the test, diagnostics process or evaluation of the result.

Most POCD self-testing-kits involve collection and performing diagnostics using the subject's (i.e., patient's or user's) urine, blood, blood plasma, tissue sample, or saliva. For example, POCD kits can be used for performing blood sugar level tests for diabetes, HIV blood tests, urine pregnancy and ovulating tests, drug tests, and many others. These kits can be safely used by the patients at home, and do not require transmission of the collected bodily fluids or tissues to the lab for testing.

More recently, with COVID-19 pandemic overwhelming the medical and lab facilities in many countries, the POCD COVED-19 antibodies blood tests were developed, which can immediately test the subject's blood for presence of COVED-19 antibodies and determine whether the subject has developed immunity to the virus. The immune response can be developed either naturally, as a natural immune response to the disease, or by having a vaccine administered to the subject.

A COVID-19 (or SARS-CoV-2) antibodies blood test determines the presence and amount of antibodies in the blood, which is inversely correlated to the probability of developing symptoms once infected by the pathogen. Over a certain threshold of antibody count an individual is considered immune to the pathogen, which confirms that the subject may be safe (or at a lower risk) for travelling, working in a closed area, or generally has a lower risk for either becoming infected with the virus, developing more dangerous or life-threatening symptoms, and/or less likely to spread the virus to others. Among other recent developments, in September of 2020, FDA issued an emergency use authorization for a Coronavirus antibody test by Assure, which can be used for self-testing in the convenience of your home, and does not require collection and transmission of the collected blood samples to the laboratory for processing and evaluation.

Among others, Abbott has developed a BINAXNOW Covid-19 Antigen self test, which is a lateral flow immunoassay for the qualitative detection of the nucleocapsid protein antigen to SARS-CoV-2, which has also been approved by FDA for self-testing in the convenience of one's home. The BINAXNOW Covid-19 home test provides a rapid and convenient method to detect the SARS-CoV-2 NP antigen based on a fluorescence immunochromatographic (FIC) assay.

One problem with POCD test that are known and used today is that they do not securely confirm the unique identity of the test subject (examinee), while also allowing the examinee to maintain anonymity, if anonymity is desired. For example, an individual who completed an HIV blood test using a home-care kit has no way to prove that he or she was the one that actually took the test.

After the outbreak of the Covid-19 pandemic, the need for a secured identity verification of the examinee person has become even more necessary. For example, a business, a store, an event, or a workplace and the like, may refrain from opening to public and in some cases to employees, in order to avoid transmission and spread of the SARS-CoV-2 virus to others, as they are unable to verify the carriers of the virus and persons who have higher chance of spreading the virus to others in another example, two individuals who take a POCD HIV test before being intimate with each other can't fully confirm and verity the other person's results, and can't fully ensure their own safety without relying solely on the good faith of the other person.

Another problem with POCD assays that are currently known and in use is that while they are relatively easy to use, many still require examinees to follow several steps that may affect the tests, and often increase the possibly of an error or inaccurate result. For example, a drug test may require the examinee to collect blood, plasma or urine in a specimen container, then insert the testing device into the sample for a specified duration of time, and then wait a specific amount of time before receiving the results. The instructions and results are not always clear, and could lead to an error by the person administering the test, and possibly to the wrong or inconclusive result.

In another example of a serologic POCD, an examinee needs to prick his or her finger with a sharp object, then drip at least a certain amount of blood into the designated test slot; thereafter add a diluent solution, and finally wait for the result to appear. When the process in such self-test has many steps that need to be performed by the subject who is not a medical professional or a scientist, there is a relatively high chance of error made during the varying multiple steps of the procedure. This effects the reliability of the test results and the outcome/diagnosis.

Thus, there is a need the POCD device and method that is more automated, and has fewer discretionary or imprecise steps or procedures that need to be performed by the test subject, thereby reducing the risk of a user error during self-administration. There is also a need to provide a POCD device and method that utilize and integrate a secured identity element into the aforementioned POCD, in order to ensure test results are associated solely with the person who took the test. There is also a need to provide a POCD device and method that maintains anonymity of the test subject, but securely confirms the identity of the subject, and prevents undesired sharing of sensitive medical information with others.

### SUMMARY OF THE INVENTION

It is one object of the invention to integrate a secure identity element into a POCD device and system in order to ensure, among other benefits, that the test results are associated solely with the examinee. Among other features, the present invention utilizes a secure identification mechanism to accomplish this objective. A device, integrated with a secure identity mechanism, triggers test initiation and/or provides test results only when the identity of the examinee is fully taken.

Another objective of the present invention is to assure that the identity of the examinee does not change during the process steps of collection of the blood, blood plasma, tissue sample, urine or saliva from the test subject and/or during the actual test processing. Moreover, the test results are stored and associated with the examinee's identity information stored within the device, to ensure that the POCD results are associated exclusively with the examinee who has undergone the test, wile simultaneously maintaining privacy and anonymity of the examinee.

Another objective of the present invention is to provide a device, system and method that can be used as art “Access Certificate” device, or as “Master Key” to access restricted areas, designated as infection/contagion free zones, or clear the examinee for travel or work, or for any activities that typically have higher risk of infecting other people (like, for example, concerts, movie theaters, inside dining, cruises, etc.)

A further objective of the present invention is to provide a device, system and method that operate as an integrated POCD system that (1) includes and integrates a biometric identity mechanism as part of the subject identification mechanism. Another objective is to provide a mechanical or computer-driven system that (2) monitors and assures that the identity of the examinee, whose blood, blood plasma or other bodily liquids are being tested, does not change during the test processing and/or during the administration of the test.

Yet another objective is to provide a system and method for a POCD test system that has requires (3) fewer and more simplified (and/or automated) steps, and requires fewer actions and less control from the user, at each of the steps following the start of the diagnostics processing. For example, the system and method is initiated by the examinee's first action of placing a finger at the specific location or position of the test, after which the administration of the test, up to the diagnostic result, is automated (either mechanically or using the computerized process, or both), to the point of providing the result or diagnostics.

Still further objective is (4) to provide or include an automated or digital “Access Certificate” or verification mechanism, which can be used to enable examinees to use the device for verification of their immunity or presence of antibodies, and to provide access to the restricted areas, such as virus-free zones, clearing the person for travel or allowing the examinee to engage in any activity that has higher risk of virus transmission to other people.

Another objective is to provide a testing device where the test results remain valid for a certain amount of time, either predefined or based on calculations of the amount of antibodies measured and the known decay rate of the pathogen antibodies.

The present invention achieves one or more objectives by providing a testing device and a method for performing a diagnostic analysis on a test subject, where the device comprises a display screen; a vessel for collecting or storing a bodily liquid from the test subject; at least one area for performing a test analysis on the body liquid or tissues of the test subject; and at least one processor executing a plurality of computer instructions stored in a computer memory, causing the device to: perform identification testing on the test subject; collect and store the identification data; evaluate the resulting test analysis data performed on the bodily liquid or tissue of the test subject, to determine at least one resulting value for the test result; perform calculations to determine whether the resulting value for the test result exceeds or reaches a threshold value; display the resulting value an the display screen; and store the resulting value in a computer memory of the device, wherein the testing and evaluation of the test analysis result is done only after successfully performing the identification testing on the test subject, and wherein the identity of the test subject is not communicated by the device to any external device.

The device and method of the present invention may be done at the point-of-service location, including self-testing by a patient, either by himself or someone who is not a medical professional. The diagnostic testing and method of the present invention may be done on a bodily liquid, such as the patient's blood, blood plasma, tissue, urine or saliva sample or on a tissue sample.

The device and method of the present invention may include one or more sensors for obtaining identification data for the test subject, and wherein the device stops the test or disables display of the test result if the identification information obtained by one or more sensors do not confirm or verify the identity of the subject prior to the display of the test result. The identification data for the test subject may be collected by a digital fingerprint scanner for scanning a fingerprint of the test subject prior to the testing and evaluation.

The device and method of present invention may also utilize a pressure sensor for sensing pressure applied by the test subject's finger during the test, or an optical sensor for detecting whether the subject removed his finger from the testing area, or any other form that ensure the subject's finger is in continuous contact with the testing area, and stopping the test or disabling display of the test result if the pressure sensor does not sense pressure of the test subject's finger, or the optical sensor detect the finger is not in contact with the testing area or other methods for ensuring the subject's finger is in continuous contact with the testing area between the start of the test and the display of the test result.

The device and method of present invention may display the resulting value as a binary value that conveys whether the resulting value exceeds or does not exceed a threshold value. In addition, or alternatively, the displayed resulting value could be a non-number that is evaluated, to determine whether the test subject's results meet a specific threshold value and whether the test subject may be allowed to enter a particular geographic area or perform a specific task based on the evaluation result.

The device and method of present invention may utilize at least partially retractable needle and a housing enclosure that at least partially houses the retractable needle, wherein the device performs the test on the blood or blood plasma sample of the test subject; and the retractable needle punctures the test subject's finger to initiate blood or blood plasma extraction from the test subject for the test.

The device and method of present invention may also include a tube, enclosure or micro-chamber, separate from the vessel, which stores at least some of the extracted bodily liquid or tissue sample. The tube, enclosure or micro-chamber may contain a diluent fluid that is mixed with the collected bodily liquid or tissue sample. Alternatively the diluent may be stored separately, and may be mixed in a separate area, chamber or enclosure. In another variation the bodily liquid or tissue may be stored in the same area (vessel, chamber, enclosure, etc.) where they are mixed with the diluent. In a further variation, the bodily liquid or tissue are mixed with the diluent in the area where the diluent is stored. In yet another variation, the bodily liquid or tissue and the diluent are all stored in separated areas, and mixed in an area that is separate from the areas where the bodily liquid or tissue, and the diluent are stored.

The present invention may be used to perform a serology test on the blood or plasma of the patient, and determines presence or concentration of antibodies to SARS-CoV2 virus or its variants. The presence or concentration of antibodies to SARS-COV2 virus or its variants may be determined at least partially based upon a presence or concentration of an IgM antibodies, an IgG antibodies, an IgA antibodies, or a combination of either one of the IgM. IgG and IgA antibodies, in the blood or plasma of the test subject.

The device and method of present invention may assign a digital time stamp to the test result stored in the computer memory of the device, wherein the digital time stamp indicates a date and time when the test was done on the test subject or when the test result may expire (either by a predefined duration from testing or by calculations based on antibodies results count and the known decay of antibodies in the immune system for the pathogen. The expiration date may be calculated by the processor, based on count of antibodies and known decay rate of the antibodies to the pathogen.

The device and method of present invention may also include an automatic locking mechanism that locks the device to prevent tempering, modifications, or re-testing.

These and other beneficial features and advantages of the present invention are disclosed in detail hereinafter, and will become more apparent upon reading the following detailed description in conjunction with the accompanying drawings, in which:

### BRIEF DESCRIPTION OF THE DRAWINGS

FIGS. 1A-B illustrate a general sequence of COVID-19 infection and development of antibodies to COVID-19 as part of an immune system response.

FIG. 1C illustrates the presence and reduction of the IgG and IgM antibodies to COVID-19 in the blood of the test subject over time.

FIG. 2 illustrates general steps of operation of a serology test that may be utilized in at least one embodiment of the present invention.

FIG. 3 illustrates a general structure and components of a POCD device that tests for COVID-19 antibodies in accordance with at least one embodiment of the present invention.

FIG. 4 illustrates process steps for using a POCD device that tests for COVID-19 antibodies in accordance with at least one embodiment of the present invention.

FIGS. 5 illustrates a process for identification of the a test subject and assuring that the identity of the test subject and the validity of the test sample is maintained from the start to the end of the test process in accordance with at least one embodiment of the present invention.

### DETAILED DESCRIPTION OF THE INVENTION

Although particularly well-suited and intended for use with COVID-19 antibody POCD tests, the present invention is not be limited to any particular application or pathogens, and the principles and specific implementation described below for a specific test kit or application are intended to be applied and used for any other POCD test where the identity of test subject needs to be confirmed, while maintaining anonymity about the test subject and not sharing the examinee's private medical and sensitive personal information with others. Thus, for the purpose of illustration only, while not limiting the invention to a specific POCD, the principles of the invention will be demonstrated over a serologic antibodies COVID-19 assay examples.

The “point-of-care diagnostics” of the present invention include diagnostics that may be done by a medical professional at an medical facility, but without the need to send the collected samples to a laboratory for analysis. It also includes the self-administered tests that allow the patient or another non-medical person to administer the diagnostics test at any location, including patient's home.

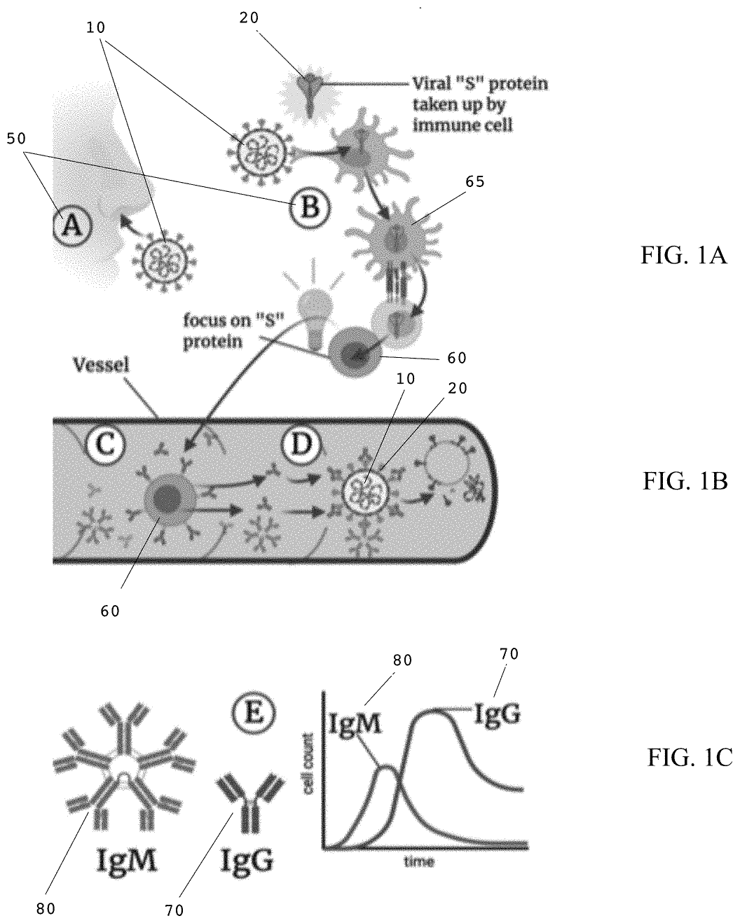

The general operation and the structure of a point-of-care diagnostic test device or a kit that is designed to identify a patient who has antibodies to SARS-CoV2 virus in the blood stream is illustrated with reference to FIGS. 1-5, The SARS-CoV2 antibodies are Y-shaped proteins 40 produced by a person's immune system to off infection. The antibodies can be naturally generated by the person's own immune system in response to the actual infection, or can be triggered by a vaccine. A correctly performed positive serology test is designed to identify patients previously infected by the SARS-CoV2 virus and/or persons who have received a vaccine, which triggers the immune response that generates antibodies in the subject's blood stream.

From the initial SARS-CoV2 infection to a positive serology test, the process is shown FIGS. 1A-B and discussed below. When a patient is infected by a SARS-CoV2 virus, the virus 10 comes in contact with the patient's immune system 50 (A, B in FIG. 1A). The key immune cells identify SARS-CoV2 as a threat to the body and absorb and display specific information about the virus. In the case of SARS-CoV2, the “S” protein 20 present on the virus exterior is most commonly identified, but other components can be identified as well. This information is passed to B cells 60, which are the key cells that produce targeted antibodies (shown as B, C in FIGS. 1A and 1B). Antibodies are the body's best defense against infection. Anti-SARS-CoV2 antibodies are Y-shaped proteins, where the arms of the Y are specifically designed to bind to the external proteins, such as the “S” protein 20 of SARS-CoV2 virus. Once these antibodies are produced, they circulate in the body, attaching to every virus they find. When the virus is covered by antibodies, the body activates the immune system mechanisms to destroy the identified SARS-CoV2 viruses (D in FIG. 1B). The so-call antigen-presenting cells or APCs 65, are the cells that can “present” antigen in a form that the T cells can recognize and attack. Among the APCs are B cells and cells of the monocyte lineage, including macrophages.

There tare also different types of antibodies. When a patient is initially infected with COVID-19 virus, B cells produce a specific kind of antibody known as IgM antibodies 80. These antibodies roam in packs of five at a time and do not last long in the body over time. FIG. 1C (E step) illustrates the decreasing presence of the IgM antibodies over time. With a passage of time, the IgM antibodies are replaced by IgG antibodies 70, which roam the body on their own and are more specific and last much longer. IgG antibodies remain in the body for a more sustained period of time, and are the key to building, immunity against the virus. The presence of the different antibodies acts as a timeline at infection: IgM indicates a recent infection, followed by both IgG and IgM presence, and lastly only or mainly IgG presence, as illustrated in FIG. 1C. However, at this time scientists are not sure how long this timeline is, or the length of time for which the presence of the specific antibodies will provide an immunity to reinfection (or the extent of this immunity). Moreover, different countries, states, geographic areas or organizations may impose different limits in connection with the types of permissions, or regulation that are permitted to or required of the test subject based on the test results.

The process of developing antibodies against a specific virus is known as seroconversion. A patient who has no antibodies against the virus in the blood serum is seronegative, while a patient with antibodies in the serum is seropositive. The COVID-19 serology test determines if the patient has undergone seroconversion. The majority of approved serology tests that are intended for the POCD use are often called the lateral flow immunoassays (LSIs) and utilize a lateral flow device (LFD). LFDs utilize a physical principle called capillarity: where liquids flow in narrow spaces without needing energy. This principle is common in the laboratory research and is also used in the home pregnancy tests.

The COVID-19 serology test uses capillarity to visually represent whether patients blood sample contains antibodies specific to the components of the SARS-CoV2 such as the “S” protein or “N” protein. The test represents this through color, although fluorescence and magnetism can also be used in other tests.

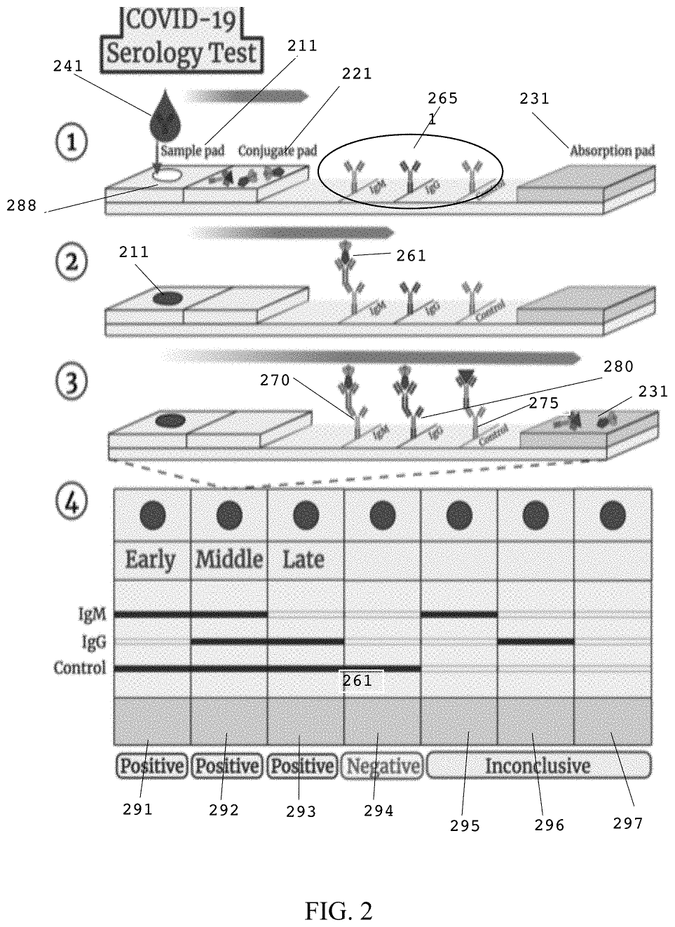

Referring to FIG. 2, the general serology test starts by placing the patient's blood sample 241 at one end of the test strip (1). The test strip contains one or more pads, including a sample pad 211 at one end of the strip, which soaks up the blood sample and allows it to flow from one end of the strip to the other 231 via capillary action. At the beginning of the strip, the conjugate pad 221 contains pieces of SARS-CoV2 “S” protein attached to the colored probes. If the patient's blood sample contains SARS-CoV2 antibodies, they will attach to these “S” protein color probes in the region 265. The sample moves down the strip with the colored “S” protein attached to any SARS-CoV2 antibodies and then meets a row 261 designed to detect antibodies 270. If the patient sample contains SARS-CoV2 IgM antibodies, these antibodies will be stopped in this region and the color from the attached “S” protein will become visible as a line on the strip (2). Next, the sample comes across a row to detect IgG antibodies 280, which works in the same manner as IgM. Lastly, a control row detects the control probes that have moved with the patient sample throughout the test. The control line 275 confirms that the test worked correctly (3). The order of IgM and IgG may vary.

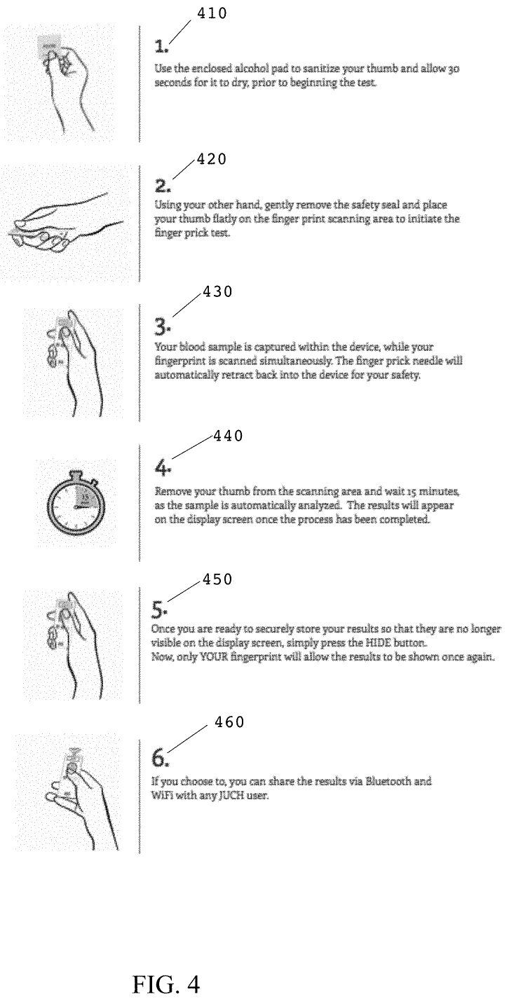

After the sample has moved, across the entire strip, the results can be read at step (4). If the test is negative for both IgG and IgM antibodies, and the control line is colored, the patient was never exposed to SARS-CoV2. This is indicated in the 4^{th} column 294 in FIG. 2 (at step 4). If any of the antibody lines are positive, the presence of the different antibodies acts as a timeline of infection. As explained above: indicates recent infection, as indicated in the 1^{st} column 291 in FIG. 2 (at step 4) followed by presence of both IgG and IgM in the 2^{nd} column 292, and lastly only by presence of the IgG in the 3^{rd} column 293 in FIG. 4 (at step 4). However, if any of the IgG or IgM strips are positive but the control strip is not, the test is invalid as indicated in the 5^{th} and 6^{th} columns 795 and 296, respectively in FIG. 2 (at step 4). If none of the IgG, IgM or control is positive, the test is also inconclusive, as indicated in the 7^{th} column 297 in FIG. 2 (at step 4).

In order to implement the biometric identity mechanism functionality, the COVID-19 POCD in accordance with at least one embodiment of the present invention is integrated with a digital fingerprint scanner. For example, the digital fingerprint scanner 288 may be located in the serology test kit shown in FIG. 2 in the approximate area of the finger-prick element of the sample pad 211, integrated in the device. Thus, when a test subject contacts the finger prick element of the device, he or she also activates the digital fingerprint scan 288 in the same area.

In an alternative embodiment, a retina scanner is utilized instead of a digital fingerprint scanner and scans the retina of the patient to preform verification of the test subiect's identity. Alternatively, a camera may take a picture or record a video of the test subject, perform image recognition processing and determine whether the correct user or test subject is using the device. The digital camera or image sensors may also be used to track the activities of the user while he or she provides a test sample, and may also track the sample until it is inserted into the device for testing. In one or more embodiments, information about the user or test subject may be previously stored in the device, communicated by the Internet to the device or scanned from an official document.

For example, the digital camera may obtain the user's or test subject's image from a driver's license, passport or another official document and compare to the image of the actual user or test subject who is using the device and performs the test, and/or who provides the test sample. The latter embodiment may be utilized with urine, saliva or other bodily fluid tests, as well as for the blood, blood plasma and tissue sample tests.

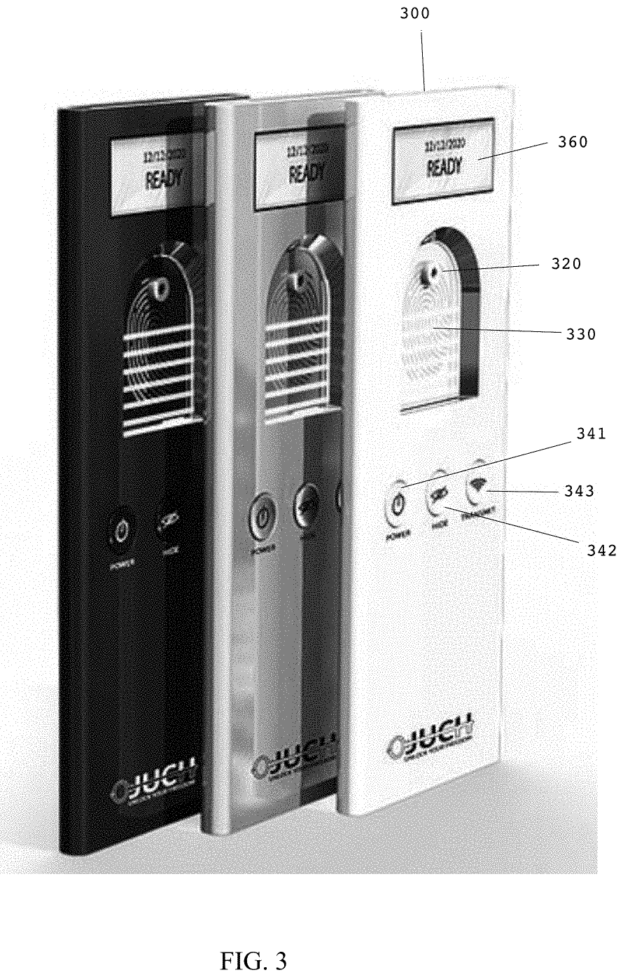

The general structure and components of a test device in accordance with at least one embodiment are shown with reference to FIG. 3 and FIG. 2. According to one embodiment of the invention, a serologic COVID-19 POCD is designed and manufactured to include, an assay device (shown in FIG. 2) for testing the presence of antibodies (or pathogens) and analysis of a given sample.

The device 300 also includes a needle (hollow or otherwise), or multiple micro-needles, which can be either fully or partially incorporated into a catheter, or multiple micro-catheter, or a lancet, or a fluid transporting tube or an other element for the purposes of pricking a tissue and transporting fluids from the exminee's tissue into the device. The hollow tube 320 in FIG. 3 may partially or fully house a needle, which extends out and pricks the finger of the test subject for blood sample collection into the opening of the tube 320. This structure further assures that the right amount of blood is collected for further processing. The device also includes a micro-chamber (not shown) containing a diluent fluid that is mixed with the collected blood or plasma of the test subject.

In at least one embodiment, the hollow tube or chamber 320 is used for housing a needle and also, at least partially used for collection of the blood within the tube or within the hollow needle. This allows the device to obtain the correct amount of blood for the test. Thus, it prevents errors that are due to insufficient or excessive amount of blood that is collected and used for mixing with diluent or during the antibody test. In at least one embodiment, when a specific amount of blood, by weight or volume is accumulated within the hollow tube or chamber 320, the needle automatically retracts, either based on a mechanical construction or by a processor executing computer instructions that cause the needle to retract back into the housing chamber or tube.

In at least one embodiment the tubes may be hollow, and may fully or partially house the needle that is used for collection of the patient's blood or blood plasma. For example, the needle can extend out of a container that may at least partially house the needle and also serve to collect the blood or blood plasma for testing. The term “retractable” refers to a mechanical action of the needle moving out of a container or chamber during the test and moving back after the test is completed.

A tube, enclosure or micro-chamber of the device may house the diluent fluid that is mixed with the collected bodily liquid or tissue sample. In at least one embodiment, the diluent fluid is stored separately from the needle enclosure and the chamber where the collected sample is stored. The diluent may then be mixed with the collected sample. In one embodiment, the mixing could be done at least partially in the same area, chamber or enclosure where the diluent is stored. In another variation, the mixing can be done in the same area (vessel, chamber, enclosure, etc.) where the sample is collected. In another embodiment, the bodily liquid or tissue and the diluent are all stored in separated areas, and are mixed in an area that is separate from the areas where the bodily liquid or tissue and the diluent are stored.

The device 300 further includes a biometric identity mechanism such as, for example, a digital fingerprint scanner 330, a computer processor and computer memory (RAM, DRAM, ROM or other internal memory for storing computer instructions that are operated upon by the processor, a power source, such as for example battier and/or DC connector for providing electricity for the operation of the device. The power can be turned on by pressing a power on/off button 341 on the device. The device can also include other operational buttons, like a “hide” button 342, for hiding the displayed results shown on a display screen, and/or a “mode” button for setting the operational mode for the device or a transmission button 343. A press of the transmission button may trigger the processor-driven functionality that causes the device to transmit the results to another device through either the WIFI Bluetooth, or using another transmitter and/or interface and communication protocol, but without transmission of the identification information about the test subject.

In at least one embodiment the device 300 includes a digital display 360, for displaying the test results and/or operational instructions and messages to the user of the device. The displayed amount can be a number that represents the antibody test result.

In at least one embodiment, the indicator can be reduced to a binary indicator, indicating “positive” value when the number or amount of the detected antibodies is greater than the set threshold. All the “negative” binary display value indicates that the number or amount of antibodies is less than the set threshold. In another embodiment, there may be also an “inconclusive” setting or display (in addition to the “positive and negative” display values). The “inconclusive” indicator indicates some range of antibodies that is not certain, according to some local regulations or company policy, to prevent the infection or to stop the person from becoming seriously ill and spreading the virus to others.

In one or more embodiments, the display displays a specific numeric value for the test result. The numeric value may be referenced and viewed on the device in order to determine, for example, the strength of the antibodies, or how much the test result is over or under the specific boundary.

The embodiment that displays the numeric value is also in for a device that may be utilized in different geographic or geo-political regions. For instance, in one geographic area, a threshold value of X1 is considered to have sufficient immunity and may permit the test subject to enter a particular area, with minimal risk to others. In another geographical area, the threshold value of X2, which is higher than X1 is considered a correct indicator of immunity 400 (or lack of pathogens). Thus, by displaying the specific numerical value, the device may indicate to the viewer whether the test subject's test results are above or below the X1 and/or X2 values. Thus, the device may be utilized in different geo-political areas or for companies or areas that implement different rules or regulations concerning the appropriate immunity or pathogen thresholds.

In at least one embodiment, the device 300 can only be activated if the finger is in full contact with the fingerprint scanner 330. Moreover, any misplacement or removal of the finger from the fingerprint scanner will immediately deactivate the device. In at least one embodiment, in the case that the plasma collection process was interrupted at any point in time during the test process, the assay is determined to be invalid or inconclusive.

In other embodiments of the invention, a retina scanner or a digital camera that performs image recognition processing may be utilized in place of a fingerprint scanner, in order to identify the biometric identity of the test subject during the test. As with a digital fingerprint, the retina scan or image recognition process confirms the identity of the test subject throughout the whole test process and/or during the bodily liquid or tissue collection process step, and any change or breaking of the contact during critical time period of the diagnostic process will render the test result invalid or inconclusive.

When utilizing a fingerprint scanner for verification, the fingerprint scanner 330 scans the finger and saves an image of the scanned fingerprint as a digital data in the device computer memory, such, for example, ROM, RAM, EPROM or other internal computer memory preferably located in the device. Alternatively, or in addition, a copy of the digital fingerprint can also be saved in the external memory. For example, the device may be equipped with a USB interface, which can be used to transmit the scanned data to an external memory of another device. The device 330 may also include a wireless communication interface, such as Bluetooth, LAN or WiFi communication transmitter and/or receiver for the wireless communications with another device and/or for the Internet connection.

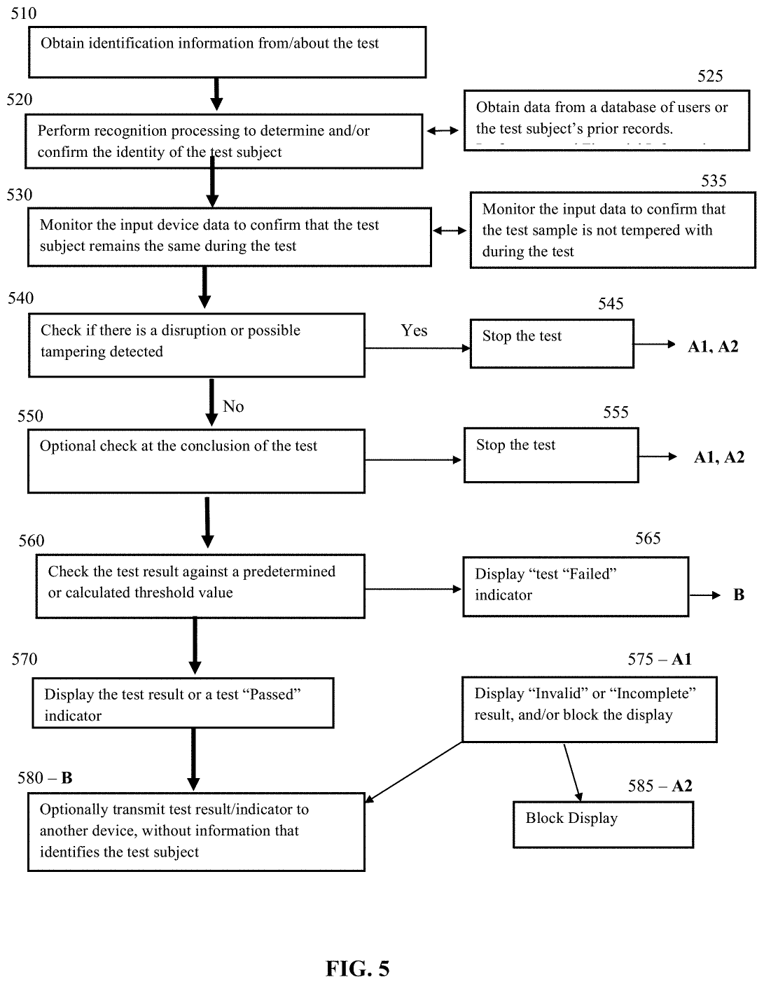

The general process for identification of the test subject and confirming that the sample provided for the test belongs to, or comes from the test subject, may also include steps to make sure that there was no tampering with the test. This is described with reference to FIG. 5.

The device and method may include one or more sensors that obtain identification information (step 510) from/about the test subject. In one embodiment, a fingerprint sensor may be used to read the digital fingerprint of the subject who is providing a blood or tissue sample for the test, and the fingerprint is taken as the subject's finger is punctured and the sample is taken. In another embodiment, an optical sensor (or multiple sensors), or a digital camera take an image of the test subject and perform recognition processing (step 520) on the obtained identification information.

In one example, the fingerprint data may be analyzed and matched against prior data in order to confirm the identity of the test subject. Alternatively, the images or video data about the test subject are compared and matched with a database or against prior records about the test subject (step 525), to confirm the identity of the test subject.

The device and system of the present invention then monitors the test subject during the test in order to confirm that the test subject remains the same person during the test (step 530). In addition, the system may also monitor the test sample, to make sure that it is not tempered with during the test (535). In one example, the system may require a constant mechanical connection of the subject's finger with the fingerprint sensor during the collection of the blood or tissue sample.

In another example, the camera or image sensor monitors the subject during the process of collection of data, and while the collected sample is applied by the device for testing/diagnostic processing. Other methods of requiring and checking for continuous contact or continuous monitoring of the test subject and the test sample are contemplated in accordance with and may be used in the aforementioned process of present invention.

In at least one embodiment, the computer processor may execute computer instructions to match the identify of the test subject and also confirm that the test sample remains the same during the collection and testing steps of the process. It may also execute computer instructions or trigger error processing if the device is tempered with or the enclosure where the test sample is placed is opened or modified during the test (step 540).

If no disruption or tempering is detected, the system may perform another optional check at the conclusion of the test, to confirm that the test subject is the same person. For example, the digital fingerprint scanner can take another reading and automatically match the digital fingerprint at the end of the test with the digital fingerprint at the start of the test, to further confirm uninterrupted processing. A video camera or image sensors may take an image and re-execute the comparison of the test subject against either the initial image the start of the test), or against the images from the database or user records (similar to step 525), or both. This step is optional and can be skipped in a least one embodiment.

If a disruption or possible tempering is detected (in step 440 and 450), the processor may execute compute instructions that make the device and system (a) stop the test (steps 545 and 555), and (b) display “Invalid” or “Incomplete” test result display message on the display of the device (step 575), and/or (c) the display of the result altogether (step 585).

If no disruption or tempering is detected (in steps 540 and 550), the processor may execute computer instructions that will cause the processor to compare the test results to some predetermined or calculated value (step 560) which determines whether the test was “passed” or failed by the test subject. If it is determined that the test was “passed”, the device may display the test result on a display screen, or display an indicator that the test was “passed” by the test subject (step 570) For example, the “passed” test for Covid-19 may indicate that the test subject's sample did not have sufficient concentration of the virus to indicate a possible infection. A “passed” test indicator for the Covid-19 antibodies may indicate that the test subject's level of immunity is above the required threshold level for a particular area or requirement.

As shown in step 580, he test result or indicator may be communicated to another device where the person may check the status of the test subject and make a decision whether the test subject can be permitted to enter a particular area or perform some work. In at least one embodiment, as part of the test result communication, the device may issue an “Access Certificate”, which could be a digital file or a print out of the result that indicates the test result status for the test subject. It might further determine whether the test subject can be permitted to enter some geographic area, cross border, perform certain work, or be permitted at certain event etc. In accordance with at least one embodiment, the “Access Certificate” may have a specific or unique digital security id, which identifies and validates the result and prevents creation of a false access certificates.

The transmission of the test result may be done through wireless network, or a proximate device-to-device communication protocol. In either case, the transmission of the test result or the indicator should not include the test subject's identification information. This will provide the requisite anonymity and safeguard privacy of the test subject, without compromising the need for a quick and efficient decision about permissions based on the test results of the administered test.

In one embodiment, if the described process utilizes a fingerprint scanner to identity and test validity confirmation, the digital data scanned by the fingerprint scanner is used for identification of the test subject. In at least one embodiment, in order to present test results on the digital display 360, the examinee is required to place his finger on the scanner for identification of the examinee. If the fingerprint of the current person does not match the stored fingerprint identification and data for the test subject, then the device will not display “positive” result, or may not display any result data at all.

In at least one embodiment, the present invention maximizes the automated processing and minimizes the steps and operations that the user must complete in order to provide a successful test/diagnostic. In at least one embodiment, the steps other than the first one, are auto-initiated, either electronically by the computer, or mechanically, or otherwise by the device rather than the subject. For example, the examinee is only required to place his finger over the scanner 330 and turn on a power switch 341.

By turning “on” the switch, the processor executes computer instructions that cause the device to perform all other steps for a complete test, from the first step to the final test result, without requiring any further actions or operations from or by the test subject. In other words, the device is equipped with the necessary mechanisms and functionality to automatically complete all the required steps, including: (1) pricking the finger of the test subject with a hollow needle, or catheter or a similar device; (2) collecting the blood or plasma in the sampling micro-cup or some chamber; (3) mixing the blood or plasma with a diluent solution; (4) testing for the existence of COVID-19 antibodies, such as IgM, IgG, or IgA; (5) displaying the test results on the display screen; and (6) storing the test data in the memory of the device.

In another example of an embodiment of the invention, these exact same elements and steps mentioned for the serologic COVIDE-19 POCD are integrated with an HIV serologic POCD.

In order to implement and allow the “Access Certificate” principle and functionality, the device holds a binary mode mechanism using a unique identifying method, reflecting positive or negative results of the test. Moreover, the binary mode results presentation is subjected to a correct biometric identification. A specific or unique id number, possibly with digital fingerprint or some other unique identifier, may be used to implement the biometric identification and “Access Certificate” principle.

The description of an exemplary process of undertaking the COVID-19 antibody test in accordance with one embodiment of the present invention is described with reference to FIG. 4. In the step 410, the examinee applies an alcohol pad to his thumb or another finger, and waits for the alcohol to dry before initiating the test. In the step 420, the examinee uses his other hand to gently remove the safety seal and place the thumb or another finger (which has been disinfected with alcohol pad) onto the fingerprint scanning area of the device. In at least one embodiment, the examinee places his finger onto the digital fingerprint scanner, located on the device in area of a pricking element. The examinee places his finger on the fingerprint scanner and turns on a switch. Turning the switch on initiates the operation and automatic performance of other steps of the test.

In the step 430, the digital fingerprint scanner scans the examinee's fingerprints for future recognition. After a predefined time interval (preferably, between 0 to 5 seconds) following completion of the scan, a concealed needle is released by the device to prick the finger. The blood is allowed to flow from the finger into the sampling pad, or micro-cup, or another collecting vessel. The sampling pad, micro chip on another collection vessel controls the amount of blood that is collected from the subject. The blood sample is captured and stored within the device the fingerprint is scanned by the digital fingerprint scanner of the device. After the pricking of the finger, the needle is automatically retracted inside the device or the collecting tube for safety reasons. The retraction can also be triggered by the processor or the mechanical construct upon the determination that the required amount of blood or plasma has been collected from the test subject and stored within the collection vessel.

After collecting the blood, a locking mechanism locks the device to prevent tempering, modifications, or re-testing in at least one embodiment, a digital time stamp (for example, indicating the exact date and time of the test) is applied. The time stamp can also be utilized by the device to trigger the retesting requirement. In at least one embodiment, the time stamp information is stored with or associated with the test result. When it is determined that the test was done over a certain period of time ago, and that the amount of antibodies may have decreased and became less effective against the, virus, the device may indicate to the user that retesting is necessary. If such retesting is necessary, the device may not display the test results unless and until the same test subject performs another test.

The time stamp, and its recorded date and time of the test, may be further utilized by the device, wherein the processor executes computer instructions to automatically calculate the expiration date for the test, based on a count of antibodies and a decay rate of the antibodies to a pathogen. In another embodiment, the test and the device may only for a single use, and must be disposed after expiration. In at least one embodiment, after expiration, the device and the display may become inactive or display “Invalid” notice instead of a test value or “Positive/Negative” (or some other binary indication).

The diluent solution, which is stored within the device, is released into the sampling pad or housing area, and mixed with the collected blood of the subject. Then, in step 410, the subject removes his or her finger from the scanning area on the pad and waits for a short period of time (for example, 15 minutes) while the sample is automatically analyzed for presence of antibodies to COVID-19. Alternatively, in another embodiment, the blood is analyzed for other viruses, conditions, and another type of test or diagnostics is performed.

In some embodiments, the diluent solution may be stored within the same enclosure or bod that houses the retractable needle. In other embodiments, the diluent solution is stored separately, and can be mixed with a blood or blood plasma sample either in another vessel or within the same vessel that houses the diluent solution. The vessel for mixing the sample with the diluent can be a third vessel, separate from those that hold the sample and the diluent.

The test results are displayed on the display screen 360 of the device. The test results may be a numeric number that represents a particular value for the test results. In at least one embodiment, the test results are displayed only for a limited time duration. In other embodiments, test result value may be assigned with a binary mode mechanism using a unique identifying method, which can be read by or shared with third parties. The binary result may indicate whether the test subject has or has no antibodies to COVID-19, has the specific amount of pathogen, or some other test condition.

The POCD device may include a unique id, which is stored in memory, and can be verified to determine that the device is produced by a reputable manufacturer and has not been improperly tempered with or misused. The chambers and the computer memory may be mechanically closed, and any attempt to access them may either disable or lock the device. Moreover, any attempt to access the computer memory of the device may disable or lock it. Other known anti-tampering methods may be utilized in accordance with present invention.

In one of the embodiments, once the examinee is ready to securely store the test results provided by the device and displayed on the display screen, he or she may press the “hide” button 342, which causes the processor to execute computer instructions that remove the displayed information from the display screen 360, and to store the test result data, together with the associated digital fingerprint data in the computer memory of the device. Alternatively, the stored results may be stored separately from the fingerprint data. For example, the data items can be stored as different records or database entries or data items, and linked or associated through a pointer or some unique identifier or the software-implemented link, map, pointer, id, etc.

In at least one embodiment, the device processor executes the software code that prevents the display of the test results in the future, unless the examinee places the finger on the fingerprint scanner and the scanned results match the stored fingerprint data. If such match occurs, the test results associated with or stored with the fingerprint data are again displayed on the display screen 360 of the device. This assured the correct verification and also provides the desired privacy and anonymity for the users of the device.

This “authorization” mode does not involve retesting. It shows the test result and the binary code (i.e., positive/negative indicator) on the display screen only when the examinees fingerprint matches the stored fingerprint data that is associated or stored with the test result that previously took place. The “authorization” mode can be invoked by a press of a “mode” button on the device and selecting the appropriate operational mode, which does not require retaking the test. The authorized results can be used as an “Access Certificate” or “Master Key” to permit the test subject to access restricted zones, obtain travel clearance or gain permission to perform any actions or be present in the areas or situations where the risk of infection is higher.

In step 460 in FIG. 4, the examinee may authorize and initiate sharing and/or transmission of the test results or the binary indicator (i.e., positive/negative) to other devices, without identification information about the user. The transmission may utilize wireless communication via WiFi, wireless LAN or Bluetooth connection and interface of the device. In another embodiment, the device could be connected via cable (for example, through a USB connection) to an external device and transmit the test results or the binary indicator through the interface to the other device, for display and verification.

In at least one embodiment, the medical data about the test subject is stored only within the device, and is not shared or made available to any third party—only the binary test result is provided to the third party or displayed on the display screen. This allows the test subject to maintain full anonymity and prevent sharing confidential, sensitive, personal and medical information with third parties or the lab.

While the examples described in this application pertain to the COVID-19 POCD, the present invention may be utilized with any POCD that uses human blood, blood plasma, tissue sample, saliva or urine sample for on-the-spot testing and/or diagnostics. In another example of an embodiment of the invention, these elements and steps mentioned for the serologic COVID-19 POCD are integrated with an HIV serologic POCD and with other POCD tests for any number of viruses, ailments or health conditions. For example, in some embodiments, the present invention can be used for urine or saliva testing, where the samples are collected from the test subject and compared for verification against prior samples, to make sure that the same subject is providing new samples for testing. In some embodiments, the camera may record the test subject while he or she is providing samples for testing and confirms that the samples come from the same subject.

It should also be understood that, although steps of various processes may be shown and described as being in a preferred sequence or temporal order, the steps of any such processes are not limited to being carried out in any particular sequence or order, absent a specific indication of such to achieve a particular intended result. In most cases, the steps of such processes may be carried in a variety of different sequences and orders, while still falling within the scope of the present inventions. In addition some steps may carried out simultaneously.

The foregoing description of the exemplary embodiments has been presented only for the purposes of illustration and description and is not intended to be exhaustive or to limit the inventions to the precise forms disclosed. Many modifications and variations are possible in light of the above teaching. Therefore, various modifications may be made without departing from the spirit and scope of the disclosure and the invention.

The embodiments were chosen and described in order to explain the principles of the inventions and their practical application so as to enable others skilled in the art to utilize the inventions and various embodiments and with various modifications as are suited to the particular use contemplated. Alternative embodiments will become apparent to those skilled in the art to which the present inventions pertain without departing from their spirit and scope.

The above embodiments and illustrative descriptions of the application of the principles of the present invention are intended to enable a person skilled in the art to make or use the disclosed invention. They are not intended to be exclusive, exhaustive or limiting on the scope of the invention described and claimed herein.

It is also understood that the invention title and abstract are not intended to limit the claimed invention or cover multiple embodiment and all various features of the claimed invention. The scope of the present inventions is defined by the appended claims rather than the foregoing description and the exemplary embodiments described therein.

I (we) hereby claim:

1. A testing device for performing a diagnostic analysis on a test subject, the device comprising:
a display screen
a vessel for collecting or storing a bodily liquid or tissue from the test subject,
at least one area for performing a test analysis on the body liquid of the test subject; and
at least one processor executing a plurality of computer instructions stored in a computer memory, causing the device to:
perform identification testing on the test subject;
collect and store the identification data;
evaluate the resulting test analysis data performed on the bodily liquid or tissue of the test subject, to determine at least one resulting value for the test result;
perform calculations to determine whether the resulting value for the test result exceeds or reaches a threshold value;
display the resulting value on the display screen; and
store the resulting value in a computer memory of the device,
wherein the testing and evaluation of the test analysis result is done only after successfully performing the identification testing on the test subject, and wherein the identity of the test subject is not communicated by the device to any external device.

2. The device of claim 1, wherein the diagnostic analysis comprises self-testing by a patient.

3. The device of claim 1, wherein the bodily liquid or tissue is a blood, blood plasma, urine, saliva or tissue sample.

4. The device of claim 1, further comprising one or more sensors for obtaining identification data for the teat subject, and wherein the device stops the test or disables display of the test result if the identification information obtained by one or more sensors does not confirm or verify the identity of the subject prior to the display of the test result.

5. The device of claim 1, wherein the identification data for the test subject is collected by a digital fingerprint scanner for scanning a fingerprint of the test subject prior to the testing and evaluation.

6. The device of claim 1, further comprising one or more sensors for determining that the test subject maintains a continuous contact with the device during the test.

7. The device of claim 1, wherein the displayed resulting value is a binary value that conveys whether the resulting value exceeds or does not exceed a threshold value.

8. The device of claim 1, wherein the displayed resulting value is a non-binary number that is evaluated, to determine whether the test subject's results meet a specific threshold value and whether the test subject may be allowed to enter a particular geographic area or perform a specific task bases on the evaluation result.

9. The device of claim further comprising:
at least partially retractable needle and a housing enclosure that at least partially houses the retractable needle, wherein the device performs the test on the blood or blood plasma sample of the test subject; and
said retractable needle punctures the test subject's finger to initiate blood or blood plasma extraction from the test subject for the test.

10. The device of claim 9, wherein the device stores at least a portion of the extracted blood or blood plasma sample in the vessel.

11. The device of claim 1, further comprising a tube, enclosure or micro-chamber, separate from the vessel that stores the extracted bodily liquid or tissue sample, said tube, enclosure or micro-chamber contains a diluent fluid that is mixed with the collected bodily fluid or tissue sample.

12. The device of claim 1, wherein the device performs a serology test on the blood or plasma of the patient, and determines presence or absence of antibodies to SARS-CoV2 virus or its variant.

13. The device of claim 12, the presence of absence of antibodies to SARS-CoV2 virus or its variant is determined at least partially based upon a presence or concentration of an IgM antibodies, an IgG antibodies, an IgA antibodies or a combination of either one of the IgM,IgG, or IgA antibodies, in the blood or plasma of the test subject.

14. The device of claim 1, wherein the processor also executes a plurality of computer instructions stored in a computer memory, causing the device to:
assign a digital time stamp to the test result stored in the computer memory of the device
wherein the digital time stamp indicates a date and time when the test was done on the test subject or when the test result may expire.

15. The device of claim 14, wherein the processor calculates an expiration date based on a count of antibodies and a decay rate of the antibodies to a pathogen.

16. The device of claim 1, further comprising a locking mechanism that locks the device to prevent tempering, modifications, or re-testing.

17. A method for performing a diagnostic analysis on a test subject, the method comprising:
collecting or storing at a bodily liquid or tissue from the test subject;
collecting and storing the identification data on the test subject;
performing identification testing on the test subject;
performing a test analysis on the bodily liquid or tissue of the test subject;
evaluating the resulting test analysis data performed on the collected bodily liquid or tissue of the test subject, to determine at least one resulting value for the test result;
performing calculations to determine whether the resulting value for the test result exceeds or reaches a threshold value;
display on a display screen the resulting value;
store the resulting value in a computer memory of the device;
wherein the testing and evaluation steps are done only after successfully performing the identification testing on the test subject, and wherein the identity of the test subject are not communicated by the device to any external device.

18. The method of claim 17, wherein the collected or stored bodily liquid or tissue is a blood, blood plasma, urine or saliva of the test subject or a tissue sample.

19. The method of claim 17, wherein the obtaining of the identification data of the test subject is performed by one or more sensors, and further comprising a step of stopping the test or disabling display of the test result if the identification information obtained by one or more sensors does not confirm or verify the identity of the subject prior to the display of the test result.

20. The method of claim 17, wherein the identification data for the test subject is collected by a digital fingerprint scanner, which scans a fingerprint of the test subject prior to the testing and evaluation.

21. The method of claim 17, further comprising: sensing pressure by a pressure sensor when applied by the test subject's finger during the test, and stopping the test or disabling display of the test result if the pressure sensor does not sense pressure of the test subject's finger between a start of the test and displaying the test result.

22. The method of claim 17, wherein the displaying test results includes displaying a binary value that conveys whether the resulting value exceeds or does not exceed a threshold value.

23. The method of claim 17, wherein the displaying test results includes displaying a non-binary number, and further includes a step of evaluating and determining whether the test subject's results meet a specific threshold value and whether the test subject may be allowed to enter a particular geographic area or perform a specific task.

24. The method of claim 17, wherein the step of collecting a bodily liquid from the test subject in performed by a retractable needle by puncturing the test subject's finger and initiating extraction of a blood or blood plasma from the test subject.

25. The method of claim 24, further comprising storing at least a portion of the extracted blood or plasma sample in the vessel.

26. The method of claim 17, further comprising: storing extracted bodily liquid or tissue sample in a tube, enclosure or micro-chamber, separate from the vessel, and mixing a diluent fluid with the collected bodily liquid or tissue sample of the patient.

27. The method of claim 17, further comprising performing a serology test on a blood or a plasma of the patient, and determining presence or absence of antibodies to SARS-COV2 virus or its variant.

28. The method of claim 27, wherein the presence or absence of antibodies to SARS-CoV2 virus or its variant is determined at least partially based upon a presence or concentration of an IgM antibodies, an IgG antibodies, or both the IgM and IgG antibodies, in the blood or plasma of the test subject.

29. The method of claim 17, further comprising:
assigning a digital time stamp to the test result stored in the computer memory of the device,
wherein the digital time stamp indicates a date and time when the test was done on the test subject or when the test result may expire.

30. The method of claim 29, further comprising automatically calculating the expiration date based on a count of antibodies and a decay rate of the antibodies to a pathogen.

31. The method of claim 17, further comprising automatically locking the device to prevent tempering, modifications, or re-testing.

37. The method of claim 17, further comprising: initiating a wired or wireless transmission of the binary form of a resulting value to another device, and providing an indication that retesting is required when the date and time of the timestamp associated with the test exceeds a specific time interval.

33. A testing device for performing a point-of-care diagnostic test for determining presence or absence of antibodies to SARS-CoV2 virus, the testing device comprising:
a display screen;
a retractable needle and a housing enclosure that houses the retractable needle;
a vessel for collecting for storing a blood or blood plasma sample collected from the test subject;
a pressure sensor for sensing pressure applied by the test subject's finger during the test, configured to stop the test disable the display if the pressure sensor does not sense pressure of the test subject's finger during the test;
a locking mechanism that locks the device to prevent tempering, modifications, or re-testing;
tube, enclosure or micro-chamber, separate from the vessel that stores the extracted blood or blood plasma sample, said tube, enclosure or micro-chamber containing a diluent fluid that is mixed with the collected blood or plasma sample;
at least one area for performing a test analysis on the collected blood or blood plasma of the test subject;
at least one processor executing a plurality of computer instructions stored in a computer memory causing the device to:
perform identification testing on the test subject, wherein the identification testing includes obtaining an identification data by scanning a digital fingerprint of the test subject by a digital scanner prior to the testing and evaluation;
collect and store the obtained identification data;
evaluate the resulting test analysis data performed on the collected blood or blood plasma of the test subject, to determine at least one resulting value for the test result;
perform calculations to determine whether the at least one resulting value for the test result exceeds or reaches a threshold value; and
display the at least one resulting value; and
store the at least one resulting value with the collected identification data for the test subject;
wherein the testing and evaluation are performed only after a successful identification of the test subject, and wherein the identity of the test subject is not communicated by the device to any external device.
